(19) United States
(12) Patent Application Publication
(10) Pub. No.: US 2025/0332102 A1
(43) Pub. Date: Oct. 30, 2025

(54) ON-DEMAND RELEASE OF ANTIBIOTIC COMPOSITION AND METHOD FOR TREATING INFECTIONS

(71) Applicant: Antinous Technology Company Limited, Macau (MO)

(72) Inventor: Kang Zhang, Macau (MO)

(21) Appl. No.: 19/192,044

(22) Filed: Apr. 28, 2025

Related U.S. Application Data
(60) Provisional application No. 63/640,133, filed on Apr. 29, 2024.

(51) Int. Cl. A61K 9/1271; A61K 31/5383; A61K 47/24; A61K 47/28; A61K 47/36; A61K 47/44; A61P 31/04

(52) U.S. Cl. CPC A61K 9/1271; A61K 31/5383; A61K 47/24; A61K 47/28; A61K 47/36; A61K 47/44; A61P 31/04

(57) ABSTRACT

Disclosed herein in some aspects are a polysaccharide-coated liposome and a composition comprising the polysaccharide-coated liposome and a pharmaceutically acceptable carrier or excipient. In some embodiments, disclosed herein is a chitosan-coated liposome with lysozyme-responsive properties for on-demand release of an antibiotic encapsulated therein in a lysozyme-rich environment while maintaining the stability of the liposome in a lysozyme-deficient environment.

### CROSS-REFERENCE TO RELATED APPLICATIONS

The present application claims priority to and benefit of U.S. Provisional Application No. 63/640,133, filed Apr. 29, 2024, the entire content of which application is incorporated herein by reference for all purposes.

### FIELD

The present disclosure relates to the field of antibacterial therapy, including stimuli-responsive drug delivery systems and therapeutic compositions.

### BACKGROUND

Conventional drugs used for antibacterial therapy display several limitations. This is not due to antibiotics being ineffective, but rather due to their low bioavailability, limited penetration to sites of infection and the rise of drug-resistant bacteria. Although new delivery systems that are loaded with antibacterial drugs have been designed to overcome these limitations, therapeutic efficacy does not seem to have improved. The present disclosure addresses these and other issues.

### BRIEF SUMMARY

Stimuli-responsive and on-demand antibiotic-loaded compositions and materials with antimicrobial properties present the ability to enhance therapeutic efficacy, while also reducing drug resistance and side effects. This promising therapeutic approach relies on advances in materials science and increased knowledge of microorganism growth and biofilm formation.

Disclosed herein in some aspects are a polysaccharide-coated liposome and a composition comprising the polysaccharide-coated liposome and a pharmaceutically acceptable carrier or excipient. In some embodiments, the polysaccharide comprises a D-glucosamine unit linked to an N-acetyl-D-glucosamine unit. In some embodiments, the polysaccharide is a chitosan. In some embodiments, the polysaccharide-coated liposome comprises an antibiotic encapsulated therein. In some embodiments, disclosed herein is composition comprises a plurality of polysaccharide-coated liposomes each independently comprising one or more different antibiotics encapsulated therein. In some embodiments, the antibiotics is levofloxacin. In some embodiments, disclosed herein is a chitosan-coated liposome with lysozyme-responsive properties for on-demand release of an antibiotic encapsulated therein in a lysozyme-rich environment while maintaining the stability of the liposome in a lysozyme-deficient environment. In some embodiments, disclosed herein is a chitosan-coated liposome with lysozyme-responsive properties for on-demand release of levofloxacin.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the polysaccharide comprises a deacetylated unit linked to an acetylated unit.

In any of the preceding embodiments, the polysaccharide can comprise a D-glucosamine unit linked to an N-acetyl-D-glucosamine unit. In any of the preceding embodiments, the polysaccharide can comprise a chitosan. In any of the preceding embodiments, the polysaccharide can have a degree of deacetylation (DD) between about 50% and about 95%.

In any of the preceding embodiments, the polysaccharide can comprise a chitosan having a degree of deacetylation (DD) of about 50%, about 55%, about 60%, about 65%, about 70%, about 75%, about 80%, about 85%, about 90%, and about 95%. In any of the preceding embodiments, the polysaccharide can comprise a chitosan having a degree of deacetylation (DD) of less than 75%, less than 60%, less than 65%, less than 60%, less than 55%, or less than 50%. In any of the preceding embodiments, the polysaccharide can be cross-linked.

In any of the preceding embodiments, the liposome can comprise a lipid bilayer comprising: i) lecithin and cholesterol, or ii) cinnamon essential oil. In any of the preceding embodiments, the weight ratio of lecithin to cholesterol in the liposome can be about 4:1. In any of the preceding embodiments, the lecithin can be soy-derived or egg-derived. In any of the preceding embodiments, the cholesterol can be synthetic or of animal origin. In any of the preceding embodiments, the liposome can be prepared using a thin-film hydration method.

In any of the preceding embodiments, the composition can comprise a plurality of polysaccharide-coated liposomes. In any of the preceding embodiments, the average particle size of the plurality of polysaccharide-coated liposomes can be between about 150 nm and about 450 nm. In any of the preceding embodiments, the average particle size of the plurality of polysaccharide-coated liposomes can be at least about 1.5, at least about 2, or at least about 3 times the average particle size of a reference plurality of liposomes which are not coated with the polysaccharide. In any of the preceding embodiments, the particle distribution index (PDI) of the plurality of polysaccharide-coated liposomes can be between about 0.6 and about 0.8. In any of the preceding embodiments, the particle distribution index (PDI) of the plurality of polysaccharide-coated liposomes can be at least about 2, at least about 3, or at least about 4 times the PDI of a reference plurality of liposomes which are not coated with the polysaccharide. In any of the preceding embodiments, the zeta potential of the plurality of polysaccharide-coated liposomes can be between about 20 mV and about 40 mV. In any of the preceding embodiments, the zeta potential of the plurality of polysaccharide-coated liposomes can be positive while the zeta potential of a reference plurality of liposomes which are not coated with the polysaccharide is negative.

In any of the preceding embodiments, the antibiotic encapsulated in the polysaccharide-coated liposome can comprise levofloxacin and/or vancomycin. In any of the preceding embodiments, the antibiotic can be present in a concentration of 1%-2% w/v in the total volume of the composition. In any of the preceding embodiments, the antibiotic can be of pharmaceutical grade with a purity of >98%. In any of the preceding embodiments, the antibiotic can be at least 5% by weight of the composition. In any of the preceding embodiments, the antibiotic can be a first antibiotic and the polysaccharide-coated liposome can further encapsulate a second antibiotic different from the first antibiotic. In any of the preceding embodiments, the polysaccharide-coated liposome can encapsulate levofloxacin and vancomycin.

In any of the preceding embodiments, the polysaccharide-coated liposome can further comprise a pharmaceutically acceptable carrier or excipient. In any of the preceding embodiments, the polysaccharide-coated liposome can further comprise a stabilizing agent. In any of the preceding embodiments, the stabilizing agent can be selected from the group consisting of glycerol, propylene glycol, and polyethylene glycol. In any of the preceding embodiments, the stabilizing agent can be present in a concentration of 0.5% w/v in the total volume of the composition. In any of the preceding embodiments, the polysaccharide-coated liposome can comprise a targeting ligand. In any of the preceding embodiments, the targeting ligand can comprise an antibody or epitope binding fragment thereof. In any of the preceding embodiments, the targeting ligand can specifically bind to Staphylococcus aureus. In any of the preceding embodiments, the targeting ligand can be on an outer surface of the polysaccharide-coated liposome. In any of the preceding embodiments, the targeting ligand can be conjugated to the polysaccharide coating of the polysaccharide-coated liposome. In any of the preceding embodiments, the polysaccharide-coated liposome can comprise an anti-fouling agent. In any of the preceding embodiments, the polysaccharide-coated liposome can comprise an agent that prevents non-specific protein adsorption. In any of the preceding embodiments, the polysaccharide-coated liposome can comprise poly(ethylene glycol) (PEG). In any of the preceding embodiments, the anti-fouling agent, the agent that prevents non-specific protein adsorption, and/or the PEG can be on an outer surface of the polysaccharide-coated liposome. In any of the preceding embodiments, the anti-fouling agent, the agent that prevents non-specific protein adsorption, and/or the PEG can be conjugated to the polysaccharide coating of the polysaccharide-coated liposome. In any of the preceding embodiments, the polysaccharide-coated liposome can comprise an antioxidant to protect the polysaccharide-coated liposome and/or the encapsulated antibiotic from degradation. In any of the preceding embodiments, the antioxidant can comprise vitamin E.

In any of the preceding embodiments, the polysaccharide-coated liposome can comprise an enzyme inhibitor to regulate the degradation rate of the polysaccharide coating of the polysaccharide-coated liposome. In any of the preceding embodiments, the enzyme inhibitor can comprise a lysozyme inhibitor. In any of the preceding embodiments, the polysaccharide-coated liposome can comprise a fluorescence marker for imaging and tracking the distribution of the liposome. In any of the preceding embodiments, the fluorescence marker can comprise fluorescein isothiocyanate (FITC).

In any of the preceding embodiments, the composition can comprise a suspension or colloid in water. In any of the preceding embodiments, the composition can be a lyophilized composition. In any of the preceding embodiments, the composition can comprise a cryoprotectant added prior to and/or after lyophilization. In any of the preceding embodiments, the cryoprotectant can comprise sucrose or trehalose.

In any of the preceding embodiments, the composition can be suitable for oral, ocular, topical, transdermal, subcutaneous, intradermal, oral, intranasal, intratracheal, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, or epicutaneous administration.

In any of the preceding embodiments, the composition can be encapsulated within a biodegradable polymer for controlled release. In any of the preceding embodiments, the biodegradable polymer can comprise polylactic acid (PLA) or polyglycolic acid (PGA).

In any of the preceding embodiments, the composition can be formulated for delayed release of the antibiotic until the polysaccharide-coated liposome contacts a lysozyme. In any of the preceding embodiments, the composition can be in contact with a biofilm. In any of the preceding embodiments, the biofilm can be a biofilm of a gram-negative bacterium. In any of the preceding embodiments, the biofilm can be a biofilm of S. aureus. In any of the preceding embodiments, the composition can be for use in treating a persistent bacterial infection in a subject in need thereof.

In some embodiments, disclosed herein is a use of a composition disclosed herein in treating a persistent bacterial infection in a subject in need thereof. In some embodiments, disclosed herein is a use of a composition disclosed herein in the manufacture of a medicament for treating a persistent bacterial infection in a subject in need thereof.

In some embodiments, disclosed herein is a method of treating a persistent bacterial infection in a subject in need thereof, comprising administering an effective amount of a composition disclosed herein to the subject. In any of the preceding embodiments, the persistent bacterial infection can comprise an S. aureus infection. In any of the preceding embodiments, the polysaccharide-coated liposome in the composition can contact a biofilm in the subject. In any of the preceding embodiments, the polysaccharide coating of the polysaccharide-coated liposome can be degraded by a lysozyme at the biofilm in the subject, thereby releasing the antibiotic at the biofilm. In any of the preceding embodiments, the polysaccharide coating of the polysaccharide-coated liposome can form a conjugate with the lysozyme at the biofilm in the subject.

### BRIEF DESCRIPTION OF DRAWINGS

The drawings illustrate certain features and advantages of this disclosure. These embodiments are not intended to limit the scope of the appended claims in any manner.

FIG. 1A shows an exemplary schematic diagram of the formation and degradation of chitosan-coated liposomes.

FIG. 1B shows an exemplary flow chart of a preparation process of chitosan-coated liposomes.

FIG. 2A shows an exemplary physical appearance of uncoated (left panel) and chitosan-coated (right panel) liposomes.

FIG. 2B shows an exemplary physical characterization of uncoated and chitosan-coated liposomes, by featuring encapsulation efficiency (EE) and drug loading (DL).

FIG. 2C shows an exemplary physical characterization of uncoated and chitosan-coated liposomes, by featuring Z-average size and particle distribution index (PDI).

FIG. 2D shows an exemplary physical characterization of uncoated and chitosan-coated liposomes, by featuring Zeta potential.

FIG. 3A shows an exemplary material characterization of uncoated and chitosan-coated liposomes, by featuring UV-vis absorption spectra.

FIG. 3B shows an exemplary material characterization of uncoated and chitosan-coated liposomes, by featuring attenuated total reflectance-Fourier transform infrared (ATR-FTIR) spectra.

FIG. 3C shows an exemplary material characterization of uncoated and chitosan-coated liposomes, by featuring X-ray diffraction analysis (XRD) spectra.

FIG. 3D shows an exemplary material characterization of uncoated and chitosan-coated liposomes, by featuring thermogravimetric analysis (TG).

FIG. 3E shows an exemplary material characterization of uncoated and chitosan-coated liposomes, by featuring derivative thermogravimetry (DTG).

FIG. 3F shows an exemplary material characterization of uncoated and chitosan-coated liposomes, by featuring differential scanning calorimetry (DSC).

FIG. 3G shows an exemplary transmission electron microscopy (TEM) photograph of the liposome containing levofloxacin without chitosan coating.

FIG. 3H shows an exemplary transmission electron microscopy (TEM) photograph of the liposome containing levofloxacin with chitosan coating.

FIG. 4A shows an exemplary release behavior of levofloxacin from levofloxacin-liposomes (Lef@Lip), encapsulated with chitosan at various degrees of deacetylation (DD), catalyzed by lysozyme at a concentration of 1.25 mg/mL.

FIG. 4B shows an exemplary release behavior by the concentration of reducing sugar produced from Lef@Lip, encapsulated with chitosan at various degrees of deacetylation (DD), catalyzed by lysozyme at a concentration of 1.25 mg/mL.

FIG. 4C shows an exemplary diagram of the active center of lysozyme and the action site of chitosan hydrolysis.

FIG. 4D shows an exemplary release behavior of levofloxacin from Lef@Lip, encapsulated with chitosan at various degrees of deacetylation (DD), catalyzed by lysozyme at various concentrations of 0, 0.5, 1, 2, and 4 mg/mL.

FIG. 5A shows an exemplary SEM image of surface morphology of chitosan-coated levofloxacin-liposomes (Lef@Lip@CS) without enzymatic hydrolysis at 5,000× magnification.

FIG. 5B shows an exemplary SEM image of surface morphology of Lef@Lip@CS without enzymatic hydrolysis at 10,000× magnification.

FIG. 5C shows an exemplary SEM image of surface morphology of Lef@Lip@CS with enzymatic hydrolysis (lysozyme concentration=1.25 mg/mL) at 5,000× magnification.

FIG. 5D shows an exemplary SEM image of surface morphology of Lef@Lip@CS with enzymatic hydrolysis (lysozyme concentration=1.25 mg/mL) at 10,000× magnification.

FIG. 6A shows an exemplary in vitro antibacterial effects of uncoated and chitosan-coated liposomes samples against S. aureus, by featuring inhibition zone performance of in vitro susceptibility test using agar diffusion method (A: chitosan-coated liposomes (Lip@CS), B: chitosan-coated liposomes catalyzed by lysozyme at 1.25 mg/ml (Lip@CS-E (1.25 mg/mL)), C: Lip@CS-E (2.5 mg/mL), D: chitosan-coated levofloxacin-liposomes (Lef@Lip@CS), E: chitosan-coated levofloxacin-liposomes catalyzed by lysozyme at 1.25 mg/mL Lef@Lip@CS-E (1.25 mg/mL) and F: Lef@Lip@CS-E (2.5 mg/mL).

FIG. 6B shows an exemplary in vitro antibacterial effects of uncoated and chitosan-coated liposomes samples against S. aureus, by featuring diameters of the zones of inhibition (ZOI) of the samples.

FIG. 6C shows an exemplary in vitro antibacterial effects of uncoated and chitosan-coated liposomes samples against S. aureus, by featuring growth curves of S. aureus in different samples.

FIG. 6D shows an exemplary in vitro antibacterial effects of uncoated and chitosan-coated liposomes samples against S. aureus, by featuring bacterial viability of S. aureus after 6-hour incubation at 37° C. with different samples.

FIG. 6E shows an exemplary in vitro antibacterial effects of uncoated and chitosan-coated liposomes samples against S. aureus, by featuring photographs of the culture media taken 24 hours after inoculation at 37° C. with treated and diluted suspension of S. aureus.

FIG. 6F shows an exemplary in vitro antibacterial effects of uncoated and chitosan-coated liposomes samples against S. aureus, by featuring colony-forming unit (CFU) of S. aureus on the agar plates counted after 24-h inoculation at 37° C. with treated and diluted suspension.

FIG. 6G shows an exemplary in vitro antibacterial effects of uncoated and chitosan-coated liposomes samples against S. aureus, by featuring photographs of crystal violet stained S. aureus biofilms treated with the different samples.

FIG. 6H shows an exemplary in vitro antibacterial effects of uncoated and chitosan-coated liposomes samples against S. aureus, by featuring corresponding absorbance at 570 nm of photographs of crystal violet stained S. aureus biofilms treated with the different samples.

FIG. 7 shows an exemplary cell viability of HepG2 cells after incubation with different concentrations of chitosan-coated liposomes (Lip@CS) for 24 h.

### DETAILED DESCRIPTION

The disclosures of all publications, patents, patent applications and published patent applications referred to herein are hereby incorporated herein by reference in their entirety. All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “mammal” encompasses both humans and non-humans and includes but is not limited to humans, non-human primates, canines, felines, murines, bovines, equines, and porcines.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. The methods of the application contemplate any one or more of these aspects of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and t e appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

Liposomes are spherical lipid vesicles (usually 50-500 nm in diameter particle size) composed of one or more lipid bilayers, as a result of emulsifying natural or synthetic lipids in an aqueous medium. In some embodiments, disclosed herein is a liposome coated with a polysaccharide. In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the polysaccharide comprises a deacetylated unit linked to an acetylated unit. In some embodiments, the polysaccharide comprises a D-glucosamine unit linked to an N-acetyl-D-glucosamine unit. In some embodiments, the polysaccharide comprises a chitosan. In some embodiments, the polysaccharide has a degree of deacetylation (DD) between about 50% and about 95%. In some embodiments, the polysaccharide is a chitosan having a degree of deacetylation (DD) of about 50%, about 55%, about 60%, about 65%, about 70%, about 75%, about 80%, about 85%, about 90%, and about 95%. In some embodiments, the polysaccharide is a chitosan having a degree of deacetylation (DD) of less than 75%, less than 60%, less than 65%, less than 60%, less than 55%, or less than 50%. In some embodiments, the polysaccharide is cross-linked.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the liposome comprises a lipid bilayer comprising: i) lecithin and cholesterol, or ii) cinnamon essential oil. In some embodiments, the weight ratio of lecithin to cholesterol in the liposome is about 4:1. In some embodiments, the lecithin is soy-derived or egg-derived, and wherein the cholesterol is synthetic or of animal origin.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the liposome is prepared using a thin-film hydration method.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition comprises a plurality of polysaccharide-coated liposomes. In some embodiments, the average particle size of the plurality of polysaccharide-coated liposomes is between about 150 nm and about 450 nm. In some embodiments, the average particle size of the plurality of polysaccharide-coated liposomes is at least about 1.5, at least about 2, or at least about 3 times the average particle size of a reference plurality of liposomes which are not coated with the polysaccharide. In some embodiments, the particle distribution index (PDI) of the plurality of polysaccharide-coated liposomes is between about 0.6 and about 0.8. In some embodiments, the particle distribution index (PDI) of the plurality of polysaccharide-coated liposomes is at least about 2, at least about 3, or at least about 4 times the PDI of a reference plurality of liposomes which are not coated with the polysaccharide. In some embodiments, the zeta potential of the plurality of polysaccharide-coated liposomes is between about 20 mV and about 40 mV. In some embodiments, the zeta potential of the plurality of polysaccharide-coated liposomes is positive and the zeta potential of a reference plurality of liposomes which are not coated with the polysaccharide is negative.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the antibiotic encapsulated in the polysaccharide-coated liposome is levofloxacin or vancomycin. In some embodiments, the antibiotic is present in a concentration of 1%-2% w/v in the total volume of the composition. In some embodiments, the antibiotic is of pharmaceutical grade with a purity of >98%. In some embodiments, the antibiotic is at least 5% by weight of the composition. In some embodiments, the antibiotic is a first antibiotic and the polysaccharide-coated liposome further encapsulates a second antibiotic different from the first antibiotic. In some embodiments, the polysaccharide-coated liposome encapsulates levofloxacin and vancomycin.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition further comprises a pharmaceutically acceptable carrier or excipient. In some embodiments, the composition further comprises a stabilizing agent. In some embodiments the stabilizing agent is selected from the group consisting of glycerol, propylene glycol, and polyethylene glycol. In some embodiments, the stabilizing agent is present in a concentration of 0.5% w/v in the total volume of the composition.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the polysaccharide-coated liposome comprises a targeting ligand. In some embodiments, the targeting ligand is an antibody or epitope binding fragment thereof. In some embodiments, the targeting ligand specifically binds to Staphylococcus aureus. In some embodiments, the targeting ligand is on an outer surface of the polysaccharide-coated liposome. In some embodiments, the targeting ligand is conjugated to the polysaccharide coating of the polysaccharide-coated liposome.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition further comprises an anti-fouling agent. In some embodiments, the composition further comprises an agent that prevents non-specific protein adsorption. In some embodiments, the composition further comprises poly(ethylene glycol) (PEG). In some embodiments, the anti-fouling agent, the agent that prevents non-specific protein adsorption, and/or the PEG is on an outer surface of the polysaccharide-coated liposome. In some embodiments, the anti-fouling agent, the agent that prevents non-specific protein adsorption, and/or the PEG is conjugated to the polysaccharide coating of the polysaccharide-coated liposome.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition further comprises an antioxidant to protect the polysaccharide-coated liposome and/or the encapsulated antibiotic from degradation. In some embodiments, the antioxidant is vitamin E. In some embodiments, the composition further comprises an enzyme inhibitor to regulate the degradation rate of the polysaccharide coating of the polysaccharide-coated liposome. In some embodiments, the enzyme inhibitor is a lysozyme inhibitor. In some embodiments, the composition further comprises a fluorescence marker for imaging and tracking the distribution of the liposome, optionally wherein the fluorescence marker is fluorescein isothiocyanate (FITC). In some embodiments, the composition further comprises a cryoprotectant added prior to and/or after lyophilization. In some embodiments, the cryoprotectant is sucrose or trehalose.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition is a suspension or colloid in water. In some embodiments, the composition is a lyophilized composition. In some embodiments, the composition is suitable for oral, ocular, topical, transdermal, subcutaneous, intradermal, oral, intranasal, intratracheal, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, or epicutaneous administration. In some embodiments, the composition is encapsulated within a biodegradable polymer for controlled release. In some embodiments, the biodegradable polymer is polylactic acid (PLA) or polyglycolic acid (PGA). In some embodiments, the composition is formulated for delayed release of the antibiotic until the polysaccharide-coated liposome contacts a lysozyme.

In some embodiments, disclosed herein is a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the composition is in contact with a biofilm. In some embodiments, the biofilm is a biofilm of a gram-negative bacterium. In some embodiments, the biofilm is a biofilm of S. aureus. In some embodiments, the composition is for use in treating a persistent bacterial infection in a subject in need thereof. In some embodiments, the composition is in treating a persistent bacterial infection in a subject in need thereof. In some embodiments, the composition is a medicament for treating a persistent bacterial infection in a subject in need thereof.

In some embodiments, disclosed herein is a method of treating a persistent bacterial infection in a subject in need thereof, comprising administering an effective amount of a composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme. In some embodiments, the persistent bacterial infection is S. aureus infection. In some embodiments, the polysaccharide-coated liposome in the composition contacts a biofilm in the subject. In some embodiments, the polysaccharide coating of the polysaccharide-coated liposome is degraded by a lysozyme at the biofilm in the subject, thereby releasing the antibiotic at the biofilm. In some embodiments, the polysaccharide coating of the polysaccharide-coated liposome forms a conjugate with the lysozyme at the biofilm in the subject.

### EXAMPLES

The following examples are included for illustrative purposes only and are not intended to limit the scope of the invention.

### Example 1. Chitosan-Coated Liposome with Lysozyme-Responsive Properties for On-Demand Release of Levofloxacin

### 1. Overview

Bacterial infections present a significant challenge to global public health, with Staphylococcus aureus being the most pathogenic among several common staphylococci and capable of causing skin, lungs, heart valve, and bone infections as well as bacterial keratitis—a sight-threatening ocular disease (Tong et al., 2015; Wu et al., 2010). While antibiotics are recognized as effective drugs for treating bacterial infections and reducing mortality and morbidity rates while saving patients' lives, their lack of specificity results in low bioavailability due to their rapid metabolism and excretion by the circulatory system before reaching the site of infection. Therefore, it is frequently necessary to use large doses of antibiotics to maintain the therapeutic effect, which may cause serious side effects on other normal tissues, such as the liver and kidney. For instance, although 0.5% and 1.5% levofloxacin solutions are effective in treating acute and subacute conjunctivitis, bacterial keratitis, and keratoconjunctivitis (Gupta et al., 2015), excessive use of the medication may lead to corneal damage (Otake et al., 2021). Furthermore, overuse of antibiotics can engender bacterial resistance, undermining their clinical efficacy (Chen et al., 2018; Levy and Marshall, 2004). Drug delivery systems, such as nanoliposomes, nanogels, micelles, and solid nanoparticles, have demonstrated their ability to deliver antimicrobial drugs for treating intracellular infections. Extensive research has been conducted to enhance the bioavailability of these systems (Ameeduzzafar et al., 2018; Le-Deygen et al., 2023; Razdan et al., 2023).

Recent innovations in drug delivery systems have attempted to optimize drug release behavior by designing and constructing bio-composites with specialized structures that enable precise control over the timing and location of drug release. Enzymes are considered to be molecular switches capable of regulating the “on-demand” release of antimicrobials from various carriers (Zhou et al., 2022). This on-demand drug delivery system is referred as an enzyme-triggered smart drug delivery system. Research has focused on enzymes that trigger the release of antimicrobials produced by bacteria (including lysozymes, lipases, hyaluronidases, pectinases, and proteases). Lysozyme is ubiquitous in animal tissues and fluids including blood, skin, saliva, urine, milk, and respiratory and cervical secretions (Sarkar et al., 2020). Notably, during bacterial infections, the immune system response increases the activity levels of several enzymes (Alves et al., 2021). Particularly in chronically infected wounds, lysozyme exhibits high activity levels (Tallian et al., 2019). Moreover, intestinal pathogens disrupt cellular function, leading to considerable secretion of lysozyme in the gut to protect against bacterial invasion (Bel et al., 2017).

This example describes a lysozyme-sensitive antibiotic delivery system for targeted delivery at the site of a surge in lysozyme caused by a bacterial infection, enabling rapid control over bacterial infection. Chitosan, a biodegradable and biocompatible polysaccharide, is used as a carrier material for this purpose since it can be hydrolyzed into N-acetylglucosamine and mono- or oligosaccharides of glucosamine and then absorbed by the human body (Primo et al., 2018). A vancomycin-loaded chitosan-polyaniline microgel was developed for lysozyme-triggered vancomycin release in the specific lysozyme-rich environment of the inflamed intestine (Li et al., 2023). A chitosan-based nanoparticle loaded with timolol maleate has been developed and incorporated into contact lenses for glaucoma therapy (Kim et al., 2014). Tamoxifen-loaded chitosan nanoparticles have been used to achieve lysozyme-triggered drug release in Caco-2 cells (Barbieri et al., 2013).

Liposome technology has been used in medicine, food, cosmetics and other fields. Despite the advanced biological features of liposomes as a means of substance delivery, their applicability is restricted by their fragile phospholipid bilayer structure (Kumar et al., 2020). A viable strategy has been proposed to modify the surface properties of liposomes to enhance their applicability by coating them with polymers to create a bio adhesive layer (Rodrigues et al., 2012). For instance, negatively charged chitosan can bind with positively charged liposomes, thereby enhancing their drug-carrying capacity. Studies have shown that compared with traditional phospholipid liposomes, chitosan-coated liposomes exhibit improved thermal stability, storage stability, and curcumin release kinetics. Cell experiments have demonstrated that this combination improves the bioavailability of curcumin (Hasan et al., 2016b; Li et al., 2017a; Peng et al., 2017). Given the ability of chitosan to disrupt bacterial cell membranes (Kandimalla et al., 2013), its binding with dicloxacillin-containing liposomes facilitates cellular internalization, thereby enhancing the intracellular penetration of antibacterials and amplifying their bacteriostatic effect (Alshamsan et al., 2019). However, because of chitosan's specific enzymatic response mechanism, the structure of chitosan-encapsulated liposomes may undergo degradation in the presence of lysozyme, leading to drug release. This potential property has not been reported.

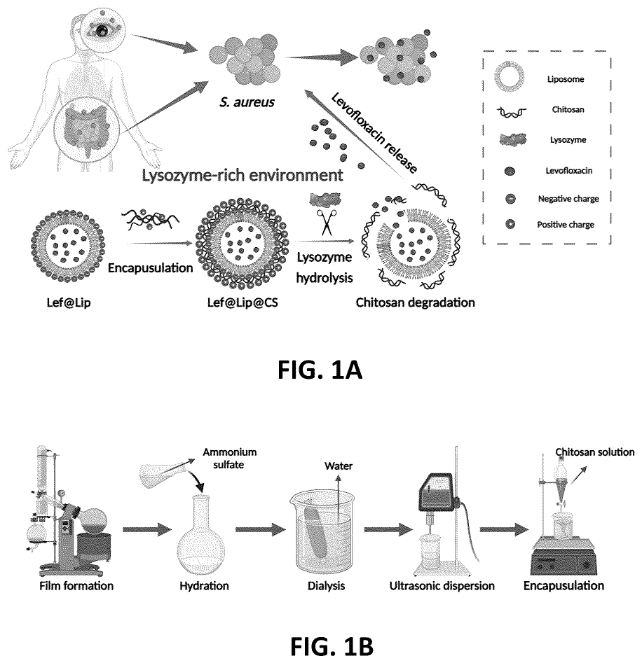

Therefore, based on the sensitivity of chitosan to degradation by lysozyme and its feasibility as an encapsulation material for liposomes, as shown in FIG. 1A, a system was proposed for the selective release of a core-shell structure based on liposomes and chitosan in response to a lysozyme-rich environment while maintaining their stability in a lysozyme-deficient environment. This study was conducted to fabricate the nanostructure of chitosan-coated levofloxacin-liposomes (Lef@Lip@CS), elucidate their physical properties through material characterization, and clarify their controlled release behavior in response to lysozyme through in vitro experiments. This system has the potential to become an effective pathway for on-demand drug release and precision anti-infection therapy.

### 2. Materials and Methods

### 2.1. Materials

Chitosan with various degrees of deacetylation (DD) (95%, 70%, and 50%), lecithin, and cholesterol were purchased from Shanghai Macklin Biochemical Co., Ltd. Levofloxacin (>98%) was purchased from Shanghai Aladdin Biochemical Technology Co., Ltd. 3-(4,5-Dimethylthiazol-2-yl)-2,5-diphenyltetrazolium bromide (MTT) and phosphate-buffered saline (PBS) were purchased from Beijing Solarbio Science & Technology Co., Ltd. Trypticase soy broth (TSB), nutrient agar, and nutrient broth medium were purchased from Guangdong Huankai Microbial Sci. & Tech. Co., Ltd. All chemical reagents were analytical grade and used without further purification. Deionized water was used in all experiments.

### 2.2. Preparation of Chitosan-Coated Levofloxacin-Liposomes

As FIG. 1B shows, the chitosan-coated liposome was prepared by the ammonium sulfate gradient method (ASGM) with minor modifications (Yan et al., 2023). First, 120 mg of lecithin and 30 mg of cholesterol were dissolved in 10 mL ethanol, and the solvent was removed from the resulting ethanol solution using a rotary evaporator at 50° C. The formed membrane was subsequently hydrated with 10 mL of 0.2 mol/L ammonium sulfate solution at 60° C. for 1 h, followed by ultrasonication in an ice bath at 200 w for 10 min with alternating cycles of ultrasound and rest using a probe ultrasonicator (each internal lasting 5 s). Then, 10 mL of the suspension of blank liposome was placed in a dialysis bag (MWCO 7 kDa) and dialyzed in 1 L of ultrapure water for 24 h. The suspension was then treated with a glacial acetic acid solution containing 1% levofloxacin (12 mg) and heated in a water bath at 60° C. for 20 min to facilitate the formation of Lef@Lip. Finally, a 0.1% (w/v) chitosan solution in acetic acid was added dropwise to the Lef@Lip suspension and magnetically stirred at 800 rpm for 24 h to form the Lef@Lip@CS.

### 2.3. Particle Size, Polydispersity Index (PDI), and Zeta Potential

The particle size, PDI, and zeta potential of Lef@Lip and Lef@Lip@CS particles were measured using a Malvern Zetasizer Nano-ZS90 (Malvern Instruments Ltd., UK). The values for each sample were determined by calculating the means of no fewer than ten independent measurements.

### 2.4. Encapsulation Efficiency (EE) and Drug Loading (DL)

The concentration of total levofloxacin was determined by dispersing 1 mL of Lef@Lip or Lef@Lip@CS suspension in 10 mL of absolute ethanol, followed by measurement using a UV-vis spectrophotometer (UV-2600 Shimadzu Co., Kyoto, Japan) at a wavelength of 294 nm. For the free levofloxacin, another suspension of 1 mL Lef@Lip or Lef@Lip@CS was transferred into the inner tube of an ultrafiltration centrifuge tube (MWCO 3 kDa) and centrifuged at 4500 r/min for 10 min. The free levofloxacin was collected in the outer tube and measured at 294 nm to determine its concentration. The EE and DL were calculated using the following equations:

$$E E ( \% ) = 1 - \frac{( {C}_{free} {V}_{free} )}{( {C}_{total} {V}_{total} )} \times 100 \qquad ( 1 )$$

$$D L ( \% ) = \frac{( {C}_{total} {V}_{total} )}{( M + {C}_{total} {V}_{total} )} \times 100 \qquad ( 2 )$$

where Cₜₒₜₐₗ represents the concentration of levofloxacin in the suspension of Lef@Lip or Lef@Lip@CS prior to ultrafiltration, C_{free} represents the concentration of Lef in the outer tube after ultrafiltration, Vₜₒₜₐₗ is 1 mL, V_{free} is the volume of ultrafiltrate, and Mis the gross mass of the liposomes.

### 2.5. UV-Vis, FT-IR and XRD

The ultraviolet absorption spectra of the samples were measured in the wavelength range of 200 to 700 nm with absolute ethanol as the reference solution.

The Fourier-transform infrared (FTIR) spectra of the samples were determined using an FTIR spectrometer (IRAFFINITY-1S, China) equipped with an attenuated total reflectance (ATR) accessory. The samples were loaded in the ATR device and measured over a scanning range from 4000 to 400 cm-1 at a resolution of 1 cm-1

The crystalline/amorphous structures of the samples were examined using an X-ray diffractometer (XRD) (Rigaku Ultima IV, Japan). Each sample was placed on the sample holder of the diffractometer. Diffractograms were recorded using Kα rays generated by Cu at diffraction angles 20 from 5° to 85° at a scanning rate of 5°/min under 40 kV/40 mA.

### 2.6. Thermal Analysis

The thermal stability of the samples was evaluated using a simultaneous thermal analyzer (STA 449 F3 Jupiter, Netzsch, Germany). Each sample, weighing 3.75 mg, was analyzed over a temperature range of 30 to 800° C. at a heating rate of 10° C./min under continuous nitrogen flow (20 mL/min).

### 2.7. TEM

The microstructures of Lef@Lip and Lef@Lip@CS particles were observed using transmission electron microscopy (TEM) (HT7820, Hitachi, Japan). A drop of the diluted sample was carefully dropped onto a 200-mesh carbon-coated copper grid and allowed to stand for 5 min. Then, the grid was negatively stained with a 2% (w/v) phosphotungstic acid solution for another 5 min before being air-dried at room temperature.

### 2.8. In Vitro Release Studies

The in vitro release behavior of levofloxacin from Lef@Lip@CS was evaluated using the dialysis technique, as previously described (Abdelrahman et al., 2021). A 1-mL sample of Lef@Lip@CS was placed in a dialysis bag (MWCO 7 kDa) containing lysozyme formulations at various concentrations (0.5, 1, 2, and 4 mg/mL). The dialysis bag was immersed in 100 mL of pH 7.4 PBS and gently stirred at 37° C. Subsequently, aliquots of the solution (3 mL each) were withdrawn at predetermined time intervals, followed by the addition of an equivalent volume of dialysis medium. The absorbance of the diluted solution was measured at a wavelength of 294 nm, and the cumulative release rate was calculated as follows:

$$Cumulative release ( \% ) = \frac{{M}_{t}}{{M}_{0}} \times 100 \% \qquad ( 3 )$$

where M₀ and Mₜ are the initial total amount and the cumulative release amount at each time interval, respectively.

### 2.9. Determination of Reducing Sugars

The degradation degree of chitosan was assessed by quantifying the concentration of reducing sugar. Initially, 0.5 g of potassium ferricyanide was dissolved in a 1-L sodium carbonate solution (0.5 mol/L) and stored in a brown bottle for later use. Subsequently, the 1-mL chitosan solution, obtained after enzymatic hydrolysis, was combined with the above 4.0-mL alkaline potassium ferricyanide solution and heated in a boiling water bath for 15 min. Following cooling and centrifuging, the concentration of reducing sugars was quantified by measuring the UV absorbance at 420 nm (4420) and calculated using the following equation:

$${A}_{420} = {A}_{1} - {A}_{0} \qquad ( 4 )$$

where A₀ and A₁ are the absorbance of the blank (PBS) and the sugar solution, respectively.

### 2.10. Scanning Electron Microscopy (SEM) Observation

The surface morphology of the samples was observed by scanning electron microscopy (SEM, TESCAN MIRA 2, Tescan Group, Czech Republic). The freeze-dried sample particles were sputtered with a thin layer of gold and then imaged at an operating voltage of 10 kV.

### 2.11. Enzyme-Triggered Antimicrobial Efficacy

The antibacterial activity of enzyme-triggered Lef@Lip@CS was determined firstly using a filter-paper-sheet diffusion assay. A bacterial suspension of S. aureus with a cell density of 106 CFU/mL at a volume of 100 μL was evenly spread onto agar plates (9 mm, 20 mL) and left to dry. Subsequently, each sample equivalent to 12.5 μg/mL of levofloxacin was applied to filter paper sheets tiled on the agar plates. The plates were incubated at 37° C. for 24 h, and the diameters of the inhibition zones were then measured.

MTT assay was employed to evaluate the bacteria viability. Bacterial cells (106 CFU/mL) were incubated with PBS, Lip@CS, Lef@Lip@CS, and Lef@Lip@CS-E (200 μg/mL) at 37° C. for 24 h, followed by three rinses with PBS. Subsequently, each well of a 96-well plate was loaded with a 100-μL bacterial suspension and 50-μL MTT solution (5 mg/mL) and incubated at 37° C. for 20 min. The dissolved formazan crystals were then obtained by adding 100 μL of dimethyl sulfoxide (DMSO), and the absorbance was measured at a wavelength of 570 nm.

The 96-well plate method was subsequently employed to demonstrate further the antimicrobial efficacy triggered by the enzyme. Each well of the 96-well plates was inoculated with 100 μL of S. aureus bacteria cells (106 CFU/mL). Then, 100 μL of PBS (as a control), Lip@CS, Lef@Lip@CS, and Lef@Lip@CS-E (200 μg/mL) were separately added to the 96-well plate and incubated at 37° C. for 6 h. The optical density at 600 nm was measured using a multifunctional microplate reader (FLUOstar Omega, BMG Labtech, Germany) to assess bacterial growth. After incubation for 6 h, a volume of 100 μL from each well was spread onto an agar plate and incubated at 37° C. for another 12 h, and the number of colony-forming units was subsequently counted.

### 2.12. Effect on Biofilms Formation

The effect of the samples on biofilm formation was assessed following a previously established protocol (Liu et al., 2019). Briefly, an overnight culture of S. aureus was diluted to a concentration of 10⁸ CFU/mL in a fresh TSB medium. Subsequently, 200 μL of the diluted bacterial suspension with four samples were inoculated in a 96-well plate. Following treatment, the bacterial cells were incubated for 48 h at 37° C. to allow biofilm formation. After incubation, the planktonic cells were removed, and the residual biofilms were gently washed three times with PBS. Each well containing biofilms was then stained with 200 μL of a 0.1% crystal violet solution for 30 min, after which the excess dye was washed off, and 200 μL of a 33% acetic acid solution was added to remove bound crystal violet from the bacterial biofilm. The absorbance at 570 nm was measured using a microplate reader.

### 2.13. Cell Culture and Viability Assay

HepG2 cells (provided by the Cell Bank of the Typical Culture Collection Committee of the Chinese Academy of Sciences.) in an exponential growth phase were seeded in 96-well plates at a density of 3×10⁵/well and treated with various concentrations of Lef@Lip for 24 h. After incubation with MTT (5 mg/mL) for 4 h, the cells were collected, and 100 μL of DMSO was added to each well before the absorbance was measured at 570 nm. Cell viability was calculated using the following equation:

$$Cell viability = \frac{{A}_{1} - {A}_{2}}{{A}_{0}} \times 100 \% \qquad ( 5 )$$

where A₁ is the absorbance of the treatment group, A₂ is the absorbance of the sample blank, and A₀ is the absorbance of the blank.

### 2.14. Statistical Analysis

Tests were performed in triplicate for each sample, and the results are presented as the mean±standard deviation (SD) for each parameter. One-way analysis of variance (ANOVA) was conducted using the GraphPad Prism 9 software. A significance level of p<0.05 was considered statistically significant. The significance values are denoted using the following symbols: * indicates p<0.05, ** indicates p<0.01, *** indicates p<0.001, and *** indicates p<0.0001.

### 3. Results and Discussion

### 3.1. Characterization of Lef@Lip and Lef@Lip@CS

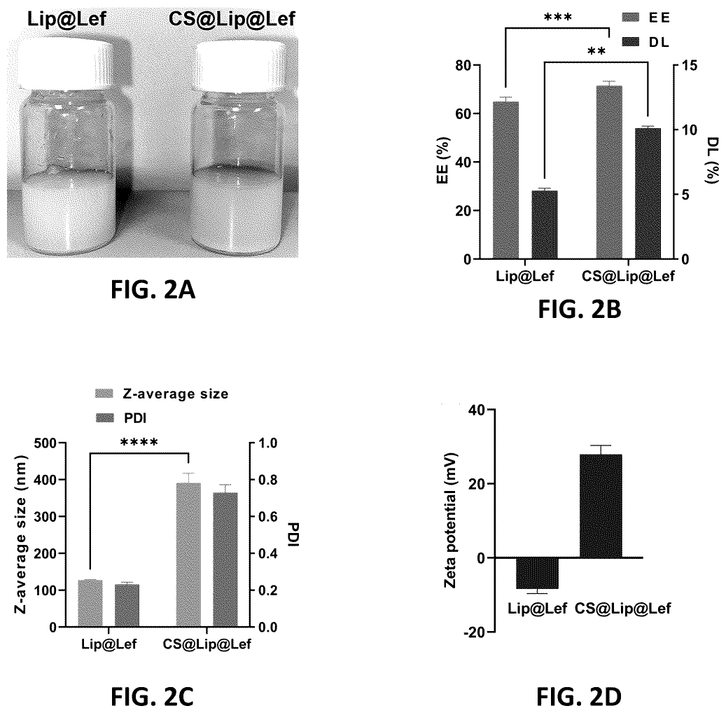

As showed in FIG. 2A, the suspensions of freshly prepared Lef@Lip and Lef@Lip@CS were translucent light blue and opalescence, respectively. As FIG. 2B showed, after chitosan coating, EE increased significantly from 64.89=1.86% to 71.43±1.89%, while DL increased from 5.28=0.18% to 10.12±0.15%. One reason for this is that, as a polymer material with good film-forming properties and abundant groups, chitosan itself can encapsulate some free levofloxacin. Another reason is that chitosan coated on the surface of the liposome or inserted into the voids of the phospholipid bilayer effectively prevents levofloxacin from leaking out of the liposomes (Li et al., 2017).

FIG. 2C showed the particle size and PDI of the Lef@Lip and Lef@Lip@CS. The uncoated liposome had an average particle size of 127.6±1.22 nm, whereas the addition of chitosan resulted in a coated liposome with an average particle size of 391.27±26.12 nm, which was approximately 3.2 times larger than its uncoated version. Because of the interaction between positive and negative charges, chitosan with a positive charge was adsorbed onto the periphery of liposomes with a negative charge. During the gradual consolidation of chitosan molecular chains, irregular coils emerged on the liposome surface, leading self-assembled aggregates of the Lip@CS system, resulting in a significant increase in particle size (Tai et al., 2020; Tan et al., 2013; Wang et al., 2021). Studies have reported similar findings. For example, Wang et al. (2021) found that chitosan modification led to an increase of approximately 500 nm in the mean particle size of cinnamaldehyde liposomes. Similarly, Tai et al. (2020) observed a significant increase in particle size with increasing chitosan concentration when encapsulating curcumin liposomes with chitosan. Dai et al. (2022) observed that the encapsulation of chitosan increased the PDI from 0.23±0.01 to 0.72±0.04, reflecting a wider particle size distribution. This may be attributable to variations in the amount of chitosan coating on the liposomes during encapsulation, leading to heterogeneous particle sizes in the final product.

The s\zeta potential is an important parameter in the characterization of the stability of liposomes. Higher surface charges (typically exceeding 30 mV in absolute value) enhance the repulsion between particles with similar charges, thereby preventing aggregation and indicating better stability (Hassane Hamadou et al., 2020). In this study, chitosan modification changed the zeta potential from −8.42±1.24 mV to 27.90±2.42 mV (FIG. 2d). The change in electrical properties demonstrated the occurrence of electrostatic connection, which served as the primary driving force behind the successful coating. Therefore, the incorporation of chitosan, in this case, effectively enhanced the system's stability (Wang et al., 2021). Similar results were obtained in a study of the liposomes of cinnamon essential oil, in which the zeta potential changed from −40.4 mV to 36.63 mV after chitosan modification (Tu et al., 2023).

### 3.2. UV-Vis, FTIR, XRD and Thermal Properties

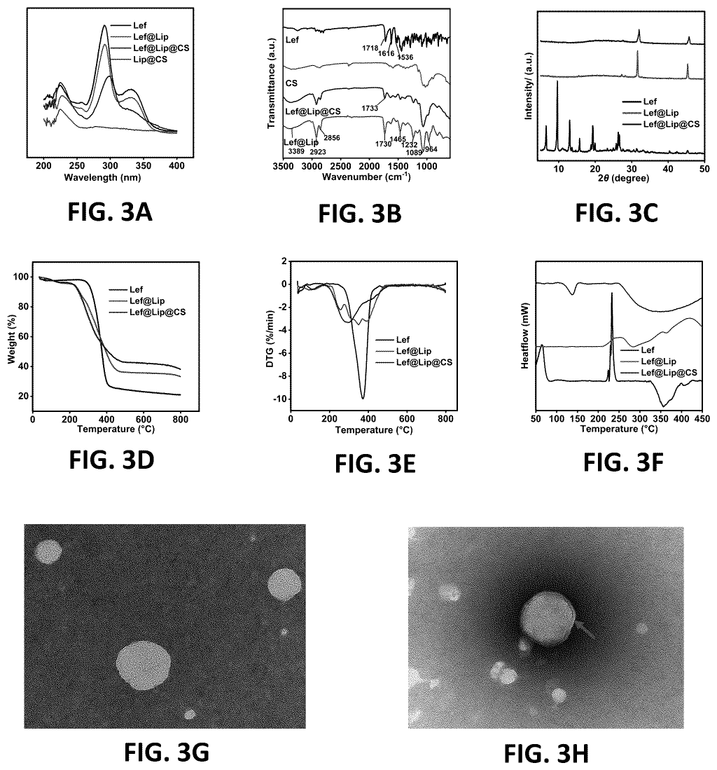

UV-Vis spectra of the samples were presented in FIG. 3A. Notably, all levofloxacin-containing samples displayed a significant absorption peak at approximately 294 nm, while the blank chitosan-coated liposome did not exhibit such a peak, suggesting that UV detection was a viable method for quantifying levofloxacin content. Furthermore, after encapsulation with chitosan, the system's absorption peak at 294 nm exhibited a slight Einstein shift, consistent with results reported previously (Hao et al., 2017).

As FIG. 3B showed, the characteristic peaks of levofloxacin (1718 cm-1, 1618 cm⁻¹, and 1536 cm⁻¹) correspond to the —COOH group, C═C group, and C—N group, respectively (Arshad et al., 2022). These distinctive peaks nearly disappeared upon encapsulation within liposomes. The Lef@Lip spectrum exhibited typical features of liposomes, including the peak at 3389 cm⁻¹ representing water vapor binding during O—H stretching and cholesterol hydroxy bond compression in the liposome structure and peaks at 2923 cm⁻¹ and 2856 cm⁻¹ representing asymmetric and symmetric stretching vibration, respectively, of C—H bonds in CH₂. Additionally, a peak at 1730 cm⁻¹ signifies C—O stretching vibration in phospholipids, a peak at 1465 cm⁻¹ represented asymmetric stretching vibration of CH₃, peaks at 1232 cm⁻¹ and 1089 cm⁻¹ indicate symmetric and asymmetric stretching vibrations of PO₂⁻, respectively, and a peak at 964 cm⁻¹ corresponded to asymmetric stretching vibration of C—C—N⁺ (Gu et al., 2022). These findings strongly suggested successful encapsulation of levofloxacin within liposomes. Furthermore, for CS-coated liposomes (Lef@Lip@CS), a significant redshift in the carbonyl absorption band toward higher frequencies (from 1730 cm⁻¹ to 1733 cm⁻¹) was observed and can be attributed to a partial negative charge on the oxygen atom, enabling it to serve as a potential binding site for chitosan conjugate. This shift indicated enhanced interactions between liposome surface and chitosan, along with reduced group hydration effects on functional groups (Hasan et al., 2016).

FIG. 3C showed the XRD patterns of levofloxacin, Lef@Lip, and Lef@Lip@CS. Sharp peaks were observed on the levofloxacin curve at several diffraction angles (6.5°, 9.7°, 12.9°, 15.7°, 19.3°, and) 26.3°, indicating its crystalline form (Gaspar et al., 2019; Gorman et al., 2012). No characteristic levofloxacin peaks were detected when levofloxacin was entrapped in liposomes or chitosan-coated liposomes, which may be because of the formation of amorphous complexes and intermolecular interactions (Liu et al., 2015). In addition, compared with Lef@Lip, the peak deformation after the addition of chitosan was wider, which may indicate the influence of chitosan encapsulation on crystallinity (Jingou et al., 2011).

FIG. 3D and FIG. 3E displayed the thermal gravity (TG) and differential thermal gravity (DTG) curves, respectively. In the temperature range of 180 to 250° C., levofloxacin, Lef@Lip, and Lef@Lip@CS experienced weight reductions of 1.8%, 4.7%, and 3.7%, respectively, presumably due to the loss of crystal water (Zhang et al., 2022). The weight loss in the range of 300 to 500° C. corresponded to a complex process involving polymer structure degradation and decomposition (Zhou et al., 2021). The uncoated levofloxacin exhibited the greatest weight loss, which decreased after layer-by-layer encapsulation. The DTG curve showed that levofloxacin did not melt rapidly after encapsulation with liposome or/and chitosan, indicating that encapsulation improved the thermal stability of the drug.

FIG. 3F showed the DSC thermograms of the samples during heating from 10 to 300° C., providing insight into the effect of temperature on the binding behavior between the encapsulation carrier and drug. The pure levofloxacin DSC thermogram showed an endothermic transition at 64.9° C. and a remarkably sharp endothermic transition peak at 232.9° C., indicating its crystalline nature, which was consistent with previous findings (El-Zahaby et al., 2014). No endothermic peak of levofloxacin was found in Lef@Lip and Lef@Lip@CS formulations, suggesting either the drug absorption into liposomes or its shift to a lower temperature, implying structural rearrangement and transition into an amorphous state (Ameeduzzafar et al., 2020; Londhe and Sharma, 2022).

The TEM images (FIG. 3G and FIG. 3H) confirmed the spheroidal morphology of both liposomes and chitosan-coated liposomes, with the latter exhibiting translucent circles (arrow) on their spherical surfaces, providing visual evidence of the formation of a shell-core structure between the outer chitosan layer and inner liposome (Song et al., 2023).

### 3.3. Levofloxacin Simulates In Vitro Release.

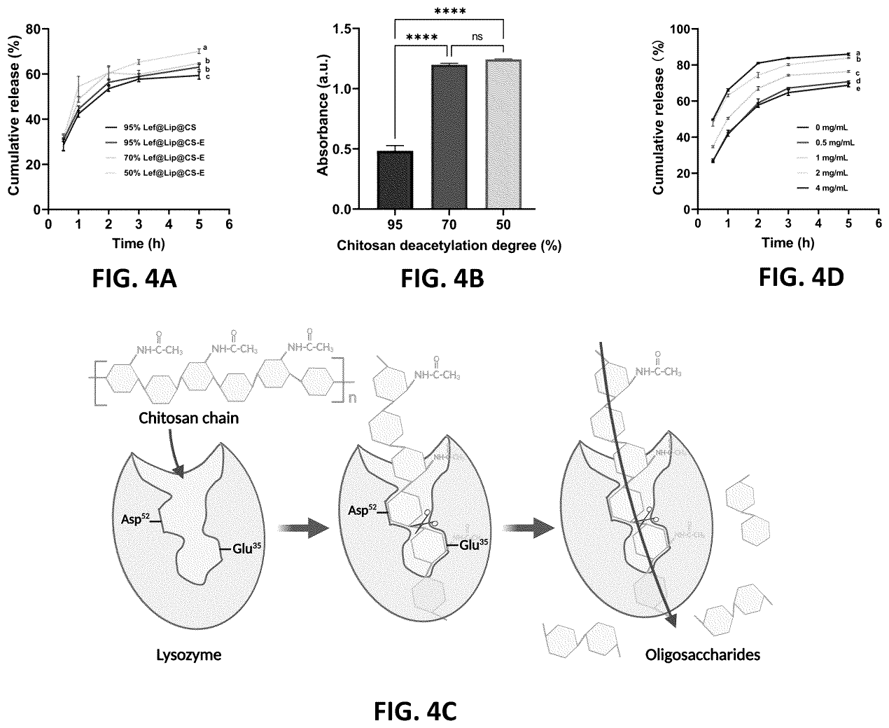

FIG. 4A illustrated the cumulative release of levofloxacin from chitosan with different DD under lysozyme catalysis. All four groups exhibited similar patterns, characterized by an initial rapid release within the first hour followed by a slow release rate after 2 h. In the absence of lysozyme, the cumulative release of levofloxacin at 5 h was approximately 59% in the 95% DD group, which was significantly increased to 63% in the presence of lysozyme. Among the groups with enzyme participation, the 50% DD group achieved the highest cumulative release of levofloxacin at 5 h, reaching approximately 70%. This finding suggested that a relatively lower DD results in enhanced lysozyme sensitivity and thereby increases the levofloxacin release rate.

The enzymatic hydrolysis of chitosan by lysozyme results in the production of reducing sugar, and thus, quantifying the concentration of reducing sugar can serve as an indicator of the extent of degradation. As FIG. 4B showed, the absorbance of solution catalyzed by lysozyme varied among chitosan samples with different DD. Notably, chitosan with a DD of 95% exhibited the lowest absorbance after the reaction, while those with DDs of 70% and 50% exhibited significantly higher absorbance levels. This observation highlighted the enhanced susceptibility to degradation under lysozyme catalysis for low-DD chitosan.

The variation in lysozyme sensitivity with DD is illustrated in FIG. 4C. The enzymatic catalysis of the substrate is facilitated by its active site, which comprises a three-dimensional space capable of accommodating the substrate and the necessary catalytic and binding groups. The crevice on the surface of lysozyme can accommodate six monosaccharide units on the polysaccharide substrate. Upon binding of the acetyl groups of chitosan with the active site's binding groups of lysozyme, catalytic groups induce conformational changes in the fourth glucose residue from a normal chair conformation to a high-energy half-chair conformation, thereby reducing glycosidic bond stability and leading to its cleavage (Song et al., 1994). However, if there is an insufficient presence of acetyl groups near the cleavage site for interaction with the active center's binding groups, the catalysis will be difficult to carry out. Consequently, higher degrees of deacetylation result in fewer acetyl groups available for substrate binding to enzymes, thus weakening enzymatic hydrolysis. This may explain why chitosan degradation by lysozyme correlates with chitosan's DD.

In addition, the effects of various concentrations of lysozyme on the release of levofloxacin were explored and found that the cumulative drug release was increased with an increasing concentration of the lysozyme. Within a span of 5 h, the maximum cumulative release (86%) was achieved at an enzyme concentration of 4 mg/mL. This could be attributed to the fact that when the substrate concentration remained constant and significantly exceeded the enzyme concentration, the enzymatic reaction rate was primarily determined by the enzyme concentration. Consequently, higher lysozyme concentrations resulted in higher degrees of chitosan degradation and levofloxacin release (FIG. 4D).

### 3.4. SEM Observed Structural Changes

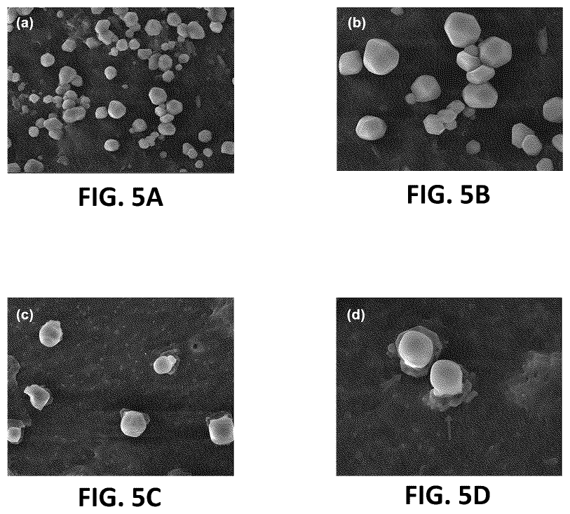

SEM was employed to examine the surface morphology of Lef@Lip@CS before and after the lysozyme hydrolysis. SEM images of Lef@Lip@CS prior to enzymolysis (FIG. 5A and FIG. 5B) exhibited spherical nanoparticles with smooth surfaces and a homogeneous size distribution, which was consistent with the typical morphology for chitosan-coated liposomes observed in previous studies (Almurshedi et al., 2021; Hao et al., 2017). Compared to the results (391.27±26.12 nm) obtained from the particle size distribution test (FIG. 2C), the SEM image revealed a larger particle diameter. This discrepancy could be attributed to the reduction of water during the lyophilization process prior to SEM analysis, leading to nanoparticle aggregation, particularly in lipid-based nano-emulsions. The decrease in solvent content disrupted the equilibrium of particle distribution and promoted self-association tendencies among nanoparticles (Diop et al., 2015; Zhong et al., 2015). Another intriguing phenomenon, illustrated in FIG. 5C and FIG. 5D, was the emergence of irregular pedestal-like structures at the bottoms of particles (indicated by red arrows). This occurrence could be attributed to the detachment of chitosan from the particle surface during lysozyme's catalytic degradation, leading to its deposition at the particle base. Some studies also confirmed this explanation. For example, Barbieri et al. (2013) demonstrated that upon co-incubation with lysozyme, the chitosan envelope of tamoxifen-loaded nanoparticles undergoes gradual decomposition and is surrounded by some enzyme proteins. Another study found that lysozyme attacks chitosan-based hydrogels to form pores of different sizes, which promote drug release (Aguanell et al., 2022).

### 3.5. Enzyme (Lysozyme)-Triggered Antibacterial Activity

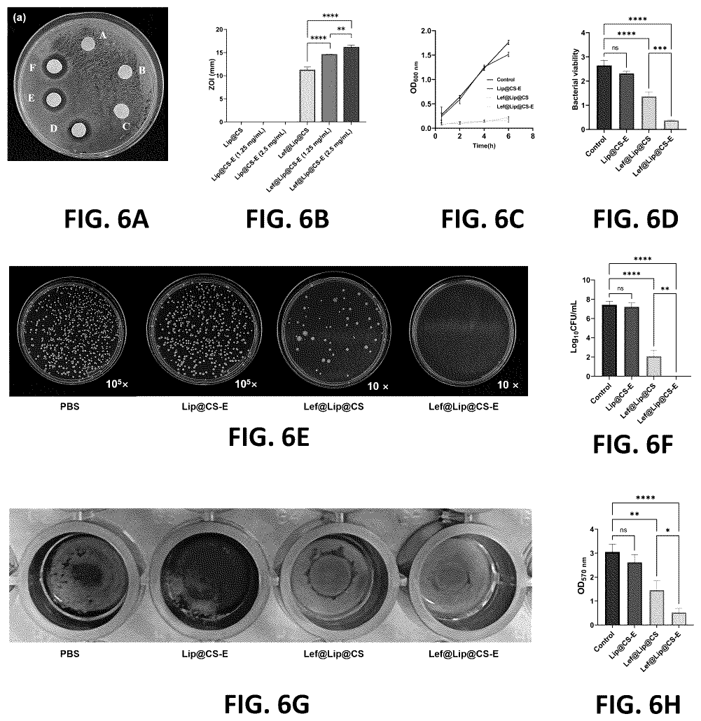

FIG. 6A to FIG. 6H showed the in vitro antibacterial effect against the gram-positive bacteria S. aureus of Lef@Lip@CS before and after enzymatic stimulation from multiple perspectives. FIG. 6A and FIG. 6B presented the results of the bacteriostatic zone of each sample on the medium. No inhibitory zone against S. aureus was observed in treatments with Lip@CS (A), Lip@CS-E (1.25 mg/mL) (B), and Lip@CS-E (2.5 mg/mL) (C). In contrast, the three levofloxacin-containing treatments Lef@Lip@CS (D), Lef@Lip@CS-E (1.25 mg/mL) (E), and Lef@Lip@CS-E (2.5 mg/mL) (F) produced clear inhibition zones, with diameters of 11.15, 14.51, and 16.06 mm, respectively. These results demonstrated the inhibitory ability of levofloxacin against S. aureus while also ruling out any potential interference from coating materials or lysozyme. Furthermore, an increase in enzyme concentration resulted in enhanced chitosan degradation and subsequent accelerated release of levofloxacin, thereby strengthening the antibacterial efficacy of the system. FIG. 6C showed the growth curve of S. aureus. Both the control group and Lip@CS with lysozyme treated group grew well within 6 h, with OD values at 6 h reaching 1.76 and 1.51, respectively; however, the Lef@Lip@CS exhibited robust antibacterial activity irrespective of the presence of lysozyme, and the OD values at 6 h were only 0.22 and 0.15, which were significantly lower than those of the two non-levofloxacin treated groups. These results also confirmed the bacteriostatic effect of levofloxacin. However, it is intriguing that Lef@Lip@CS and Lef@Lip@CS-E in this test did not exhibit statistically significant differences over the course of the 6-h duration. This outcome may be attributed to the fact that OD₆₀₀ only reflected the extent of light scattering caused by bacteria in the culture medium. The greater the bacteria population was, the more pronounced the light scattering effect became. Hence, OD₆₀₀ was associated with total bacterial count rather than specifically indicating viable bacteria (Beal et al., 2020). Therefore, the MTT test was employed to elucidate further the activation of lysozyme on the antibacterial efficacy of the system. In contrast to turbidity measurements, mitochondrial dehydrogenase in living cells metabolizes yellowish MTT to form a blue-purple precipitate, which is an effective way to detect bacterial activity (Kumar et al., 2018). As FIG. 6D showed, the bacterial viability of the control group and Lip@CS-E group were measured as 2.643 and 2.313, respectively. The disparity in results between these two groups could be attributed to the limited antibacterial properties inherent to chitosan and lysozyme themselves, which consequently influenced their respective bacterial activities. Notably, the two levofloxacin-containing groups Lef@Lip@CS and Lef@Lip@CS-E exhibited significantly reduced bacterial viability of only 1.362 and 0.36, respectively, highlighting the exceptional antibacterial properties of levofloxacin and underscoring the enhanced efficacy achieved through lysozyme supplementation. FIG. 6E and FIG. 6F illustrated the quantification of viable bacteria in the treated samples after a 6-h culture on the media. When diluted at a concentration of 10⁵× in both the control group and Lip@CS-E group, a substantial number of colonies persisted on the agar plate, with bacteria counts measuring 7.41 log and 7.01 log, respectively. Conversely, Lef@Lip@CS exhibited a bacterial count of only 2.06 log (the sterilization rate was 72.46±7.06%), while Lef@Lip@CS-E group demonstrated superior antibacterial efficacy by completely inhibiting colony formation on the agar plate, with a sterilization rate of 100% (p<0.001).

Persistent infections are invariably associated with the formation of biofilm, which is a prominent contributor to antibiotic resistance (Liu et al., 2019). Given the facile formation of biofilm by S. aureus on the surface of biomaterial, it was employed as a bacterial model to evaluate the effect of the drug delivery nano-system on biofilm inhibition. FIG. 6G and FIG. 6H presented the crystal violet staining image of the treated biofilm and the corresponding absorbance at OD₅₇₀, respectively. Clear biofilm bands were observed in the PBS and Lip@CS-E groups, firmly adhered to the pore surface, suggesting that these two treatments had minimal impact on biofilm removal. The Lef@Lip@CS group exhibited limited efficacy in removing biofilm, as evidenced by the presence of a distinct residual biofilm band and corresponding OD₅₇₀ values indicating that only approximately 51% of the biofilm was eliminated. In contrast, the Lef@Lip@CS-E group demonstrated a remarkable clearance effect triggered by the enzyme, resulting in nearly complete eradication of the biofilm. The OD₅₇₀ analysis revealed that this system removed approximately 71% of the biofilm. The extracellular polymeric substances (EPS) of a biofilm function as robust physical barriers, effectively impeding the penetration of conventional antimicrobial agents (Wang et al., 2023). Evidently, the lysozyme coordination Lef@Lip@CS system exhibits superior efficacy in clearing biofilms, which can be attributed to the enhanced release of levofloxacin. This increase in local drug concentration facilitates improved penetration and diffusion into the biofilm.

These results collectively demonstrated the synergistic effect of lysozyme on the antibacterial activity of the drug @CS system, highlighting its potential advantage in lysozyme-rich areas due to bacterial (such as S. aureus) infection. In such cases, chitosan underwent catalytic degradation, thereby accelerating the release of antibiotics within the system and achieving enhanced antibacterial efficacy. Li et al. (2023) developed a vancomycin-loaded chitosan-polyaniline microgels for lysozyme-triggered vancomycin release, specifically targeting the unique microenvironment associated with lysozyme secretion in inflamed gut conditions. The antibacterial assay revealed significantly lower viability of S. aureus in the vancomycin-loaded chitosan-polyaniline microgel (pH 6.8+ lysozyme) group compared to the vancomycin-loaded chitosan-polyaniline microgel (pH 6.8) group without lysozyme present, confirming that lysozyme-triggered chitosan degradation promotes antibiotic release, which is consistent with our experimental findings.

Another potential reason for the enhancement of antibacterial activity in the chitosan carrying system by lysozyme is its ability to form a conjugate with polysaccharides, including chitosan, as reported previously. This lysozyme-chitosan conjugate exhibits exceptional surfactant activity, which can potentially disrupt the bacterial outer membrane and cleave the peptidoglycan layer. Particularly for gram-negative bacteria, this undoubtedly augments the antimicrobial efficacy of drug delivery systems (Kim et al., 2020; Song et al., 2002).

### 3.6. Biocompatibility Evaluation

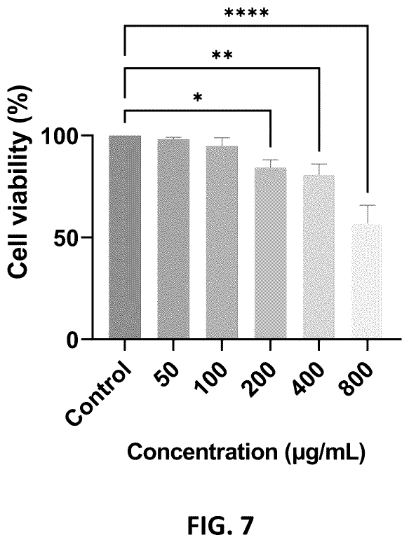

As a potential drug carrier material, it is imperative to ascertain the biocompatibility of chitosan-coated liposomes. According to GB/T 16886.5-2003 (ISO10993-5:1999), samples exhibiting cell viability exceeding 75% can be regarded as noncytotoxic (Yang et al., 2010). Moreover, for biological materials to be considered biocompatible, a minimum cell viability of 70% must be achieved (Cannella et al., 2019). The MTT test results of biomaterial-induced cytotoxicity in HepG2 cells (FIG. 7) demonstrated that at a concentration of 100 μg/mL, the cell survival rate was 90%, which did not significantly differ from that of the control group. Furthermore, at a concentration of 200 μg/mL, the cell survival rate remained above 85%. Additionally, HepG2 cells in contact with the Lip@CS exhibited normal spherical morphology and maintained proliferation throughout the culture period, with almost all cells surviving after culture. These findings unequivocally established that chitosan-coated liposomes did not have any detrimental effects on cellular integrity.

### 4. Conclusion

This study employed lysozyme-sensitive chitosan-coated liposomes as a biodegradable carrier for levofloxacin, enabling the development of a nanodrug formulation (Lef@Lip@CS) capable of on-demand antibiotic release. Comprehensive material testing and surface characterization showed that the resulting nanomaterials Lef@Lip@CS had an average particle size of 391.27 nm, EE for levofloxacin of approximately 71%, and a distinct shell-core structure. The release behavior tests demonstrated that Lef@Lip@CS ensures continuous and stable release of levofloxacin over 5 h (without enzyme). In the presence of lysozyme (1.25 mg/mL), the external chitosan coating undergoes degradation, leading to an increase in the 5-h cumulative release rate of levofloxacin from 67.77% to 85.29%. The results of bacteriostatic experiments showed that the presence of lysozyme significantly improved its antibacterial activity. Specifically, when compared to the Lef@Lip@CS group without enzyme participation, the addition of lysozyme resulted in an increase in the inhibition zone diameter from 11.15 mm to 16.06 mm, demonstrating a concentration-dependence effect. The bacterial viability decreased from 1.362 to 0.36, the sterilization rate improved from 72.46% to 100%, and the biofilm removal rate increased from 51% to 71%. The results of biocompatibility experiments showed that chitosan-coated liposomes showed good biocompatibility as drug delivery carriers. These findings indicate that the lysozyme-responsive chitosan-coated liposomes developed in this study can be expected to undergo degradation at an infection site abundant in lysozyme, thereby enhancing drug dosage. In comparison with other designed antimicrobial therapies, this specifically identified drug carrier holds significant potential to enhance antibiotic targeting delivery, achieve controlled release as needed, improve drug delivery efficiency, minimize side effects, and improve anti-infection therapy.

### REFERENCES

Abdelrahman, T. M., Qin, X., Li, D., Senosy, I. A., Mmby, M., Wan, H., Li, J., He, S., 2021. Pectinase-responsive carriers based on mesoporous silica nanoparticles for improving the translocation and fungicidal activity of prochloraz in rice plants. Chemical Engineering Journal 404, 126440. https://doi.org/10.1016/j.cej.2020.126440
Aguanell, A., del Pozo, M. L., Pérez-Martín, C., Pontes, G., Bastida, A., Fernández-Mayoralas, A., García-Junceda, E., Revuelta, J., 2022. Chitosan sulfate-lysozyme hybrid hydrogels as platforms with fine-tuned degradability and sustained inherent antibiotic and antioxidant activities. Carbohydrate Polymers 291, 119611. https://doi.org/10.1016/j.carbpol.2022.119611
Almurshedi, A. S., Aljunaidel, H. A., Alquadeib, B., Aldosari, B. N., Alfaqiah, I. M., Almarshidy, S. S., Eltahir, E. K., Mohamoud, A. Z., 2021. Development of Inhalable Nanostructured Lipid Carriers for Ciprofloxacin for Noncystic Fibrosis Bronchiectasis Treatment. IJN Volume 16, 2405-2417. https://doi.org/10.2147/IJN.S286896
Alves, P. M., Barrias, C. C., Gomes, P., Martins, M. C. L., 2021. Smart biomaterial-based systems for intrinsic stimuli-responsive chronic wound management. Materials Today Chemistry 22, 100623. https://doi.org/10.1016/j.mtchem.2021.100623
Ameeduzzafar, Alruwaili, N. K., Imam, S. S., Alotaibi, N. H., Alhakamy, N. A., Alharbi, K. S., Alshehri, S., Afzal, M., Alenezi, S. K., Bukhari, S. N. A., 2020. Formulation of Chitosan Polymeric Vesicles of Ciprofloxacin for Ocular Delivery: Box-Behnken Optimization, In Vitro Characterization, HET-CAM Irritation, and Antimicrobial Assessment. AAPS PharmSciTech 21, 167. https://doi.org/10.1208/s12249-020-01699-9
Ameeduzzafar, Imam, S. S., Abbas Bukhari, S. N., Ahmad, J., Ali, A., 2018. Formulation and optimization of levofloxacin loaded chitosan nanoparticle for ocular delivery: In-vitro characterization, ocular tolerance and antibacterial activity. International Journal of Biological Macromolecules 108, 650-659. https://doi.org/10.1016/j.ijbiomac.2017.11.170
Arshad, M. S., Kiran, M., Mudassir, J., Farhan, M., Hussain, A., Abbas, N., 2022. Formulation, Optimization, in vitro and in-vivo evaluation of levofloxacin hemihydrate Floating Tablets. Braz. J. Pharm. Sci. 58, e18630. https://doi.org/10.1590/s2175-97902022e18630
Barbieri, S., Sonvico, F., Como, C., Colombo, G., Zani, F., Buttini, F., Bettini, R., Rossi, A., Colombo, P., 2013. Lecithin/chitosan controlled release nanopreparations of tamoxifen citrate: Loading, enzyme-trigger release and cell uptake. Journal of Controlled Release 167, 276-283. https://doi.org/10.1016/j.jconrel.2013.02.009
Beal, J., Farny, N. G., Haddock-Angelli, T., Selvarajah, V., Baldwin, G. S., Buckley-Taylor, R., Gershater, M., Kiga, D., Marken, J., Sanchania, V., Sison, A., Workman, C. T., 2020. Robust estimation of bacterial cell count from optical density. Commun Biol 3, 1-29. https://doi.org/10.1038/s42003-020-01127-5
Bel, S., Pendse, M., Wang, Y., Li, Y., Ruhn, K. A., Hassell, B., Leal, T., Winter, S. E., Xavier, R. J., Hooper, L. V., 2017. Paneth cells secrete lysozyme via secretory autophagy during bacterial infection of the intestine. Science 357, 1047-1052. https://doi.org/10.1126/science.aal4677
Cannella, V., Altomare, R., Chiaramonte, G., Di Bella, S., Mira, F., Russotto, L., Pisano, P., Guercio, A., 2019. Cytotoxicity Evaluation of Endodontic Pins on L929 Cell Line. BioMed Research International 2019, e3469525. https://doi.org/10.1155/2019/3469525
Chen, M., Xie, S., Wei, J., Song, X., Ding, Z., Li, X., 2018. Antibacterial Micelles with Vancomycin-Mediated Targeting and pH/Lipase-Triggered Release of Antibiotics. ACS Appl. Mater. Interfaces 10, 36814-36823. https://doi.org/10.1021/acsami.8b16092
Dai, H., Peng, L., Wang, H., Feng, X., Ma, L., Chen, H., Yu, Y., Zhu, H., Zhang, Y., 2022. Improved properties of gelatin films involving transglutaminase cross-linking and ethanol dehydration: The self-assembly role of chitosan and montmorillonite. Food Hydrocolloids 132, 107870. https://doi.org/10.1016/j.foodhyd.2022.107870
Diop, M., Auberval, N., Viciglio, A., Langlois, A., Bietiger, W., Mura, C., Peronet, C., Bekel, A., Julien David, D., Zhao, M., Pinget, M., Jeandidier, N., Vauthier, C., Marchioni, E., Frere, Y., Sigrist, S., 2015. Design, characterisation, and bioefficiency of insulin-chitosan nanoparticles after stabilisation by freeze-drying or cross-linking. International Journal of Pharmaceutics 491, 402-408. https://doi.org/10.1016/j.ijpharm.2015.05.065
El-Zahaby, S. A., Kassem, A. A., El-Kamel, A. H., 2014. Design and evaluation of gastroretentive levofloxacin floating mini-tablets-in-capsule system for eradication of Helicobacter pylori. Saudi Pharm J 22, 570-579. https://doi.org/10.1016/j.jsps.2014.02.009
Gaspar, M. C., Pais, A. A. C. C., Sousa, J. J. S., Brillaut, J., Olivier, J.-C., 2019. Development of levofloxacin-loaded PLGA microspheres of suitable properties for sustained pulmonary release. International Journal of Pharmaceutics 556, 117-124. https://doi.org/10.1016/j.ijpharm.2018.12.005
Gorman, E. M., Samas, B., Munson, E. J., 2012. Understanding the Dehydration of Levofloxacin Hemihydrate. Journal of Pharmaceutical Sciences 101, 3319-3330. https://doi.org/10.1002/jps.23200
Gu, Y., Zhao, Z., Xue, F., Zhang, Y., 2022. Alginate-Chitosan Coated Nanoliposomes as Effective Delivery Systems for Bamboo Leaf Flavonoids: Characterization, In Vitro Release, Skin Permeation and Anti-Senescence Activity. Antioxidants 11, 1024. https://doi.org/10.3390/antiox11051024
Gupta, H., Aqil, M., Khar, R. K., Ali, A., Bhatnagar, A., Mittal, G., 2015. An alternative in situ gel-formulation of levofloxacin eye drops for prolong ocular retention. Journal of pharmacy & bioallied sciences 7, 9.
Hao, J., Guo, B., Yu, S., Zhang, W., Zhang, D., Wang, J., Wang, Y., 2017. Encapsulation of the flavonoid quercetin with chitosan-coated nano-liposomes. LWT-Food Science and Technology 85, 37-44. https://doi.org/10.1016/j.lwt.2017.06.048
Hasan, M., Ben Messaoud, G., Michaux, F., Tamayol, A., Kahn, C. J. F., Belhaj, N., Linder, M., Arab-Tehrany, E., 2016. Chitosan-coated liposomes encapsulating curcumin: study of lipid-polysaccharide interactions and nanovesicle behavior. RSC Adv. 6, 45290-45304. https://doi.org/10.1039/C6RA05574E
Hassane Hamadou, A., Huang, W.-C., Xue, C., Mao, X., 2020. Formulation of vitamin C encapsulation in marine phospholipids nanoliposomes: Characterization and stability evaluation during long term storage. LWT 127, 109439. https://doi.org/10.1016/j.lwt.2020.109439
Jingou, J., Shilei, H., Weiqi, L., Danjun, W., Tengfei, W., Yi, X., 2011. Preparation, characterization of hydrophilic and hydrophobic drug in combine loaded chitosan/cyclodextrin nanoparticles and in vitro release study. Colloids and Surfaces B: Biointerfaces 83, 103-107. https://doi.org/10.1016/j.colsurfb.2010.11.005
Kim, H.-J., Zhang, K., Moore, L., Ho, D., 2014. Diamond Nanogel-Embedded Contact Lenses Mediate Lysozyme-Dependent Therapeutic Release. ACS Nano 8, 2998-3005. https://doi.org/10.1021/nn5002968
Kim, S., Fan, J., Lee, C.-S., Lee, M., 2020. Dual Functional Lysozyme-Chitosan Conjugate for Tunable Degradation and Antibacterial Activity. ACS Appl. Bio Mater. 3, 2334-2343. https://doi.org/10.1021/acsabm.0c00087
Kumar, P., Nagarajan, A., Uchil, P. D., 2018. Analysis of Cell Viability by the MTT Assay. Cold Spring Harb Protoc 2018. https://doi.org/10.1101/pdb.prot095505
Le-Deygen, I. M., Skuredina, A. A., Mamaeva, P. V., Kolmogorov, I. M., Kudryashova, E. V., 2023. Conjugates of Chitosan with β-Cyclodextrins as Promising Carriers for the Delivery of Levofloxacin: Spectral and Microbiological Studies. Life 13, 272. https://doi.org/10.3390/life13020272
Levy, S. B., Marshall, B., 2004. Antibacterial resistance worldwide: causes, challenges and responses. Nat Med 10, S122-129. https://doi.org/10.1038/nm1145
Li, R., Deng, L., Cai, Z., Zhang, S., Wang, K., Li, L., Ding, S., Zhou, C., 2017. Liposomes coated with thiolated chitosan as drug carriers of curcumin. Materials Science and Engineering: C 80, 156-164. https://doi.org/10.1016/j.msec.2017.05.136
Li, X., Hetjens, L., Wolter, N., Li, H., Shi, X., Pich, A., 2023. Charge-reversible and biodegradable chitosan-based microgels for lysozyme-triggered release of vancomycin. Journal of Advanced Research 43, 87-96. https://doi.org/10.1016/j.jare.2022.02.014
Liu, Y., Lin, A., Liu, Jiawei, Chen, X., Zhu, X., Gong, Y., Yuan, G., Chen, L., Liu, Jie, 2019. Enzyme-Responsive Mesoporous Ruthenium for Combined Chemo-Photothermal Therapy of Drug-Resistant Bacteria. ACS Appl. Mater. Interfaces 17.
Liu, Y., Liu, D., Zhu, L., Gan, Q., Le, X., 2015. Temperature-dependent structure stability and in vitro release of chitosan-coated curcumin liposome. Food Research International 74, 97-105. https://doi.org/10.1016/j.foodres.2015.04.024
Londhe, V. Y., Sharma, S., 2022. Formulation, characterization, optimization and in-vivo evaluation of methazolamide liposomal in-situ gel for treating glaucoma. Journal of Drug Delivery Science and Technology 67, 102951. https://doi.org/10.1016/j.jddst.2021.102951
Otake, H., Goto, R., Ogata, F., Isaka, T., Kawasaki, N., Kobayakawa, S., Matsunaga, T., Nagai, N., 2021. Fixed-Combination Eye Drops Based on Fluorometholone Nanoparticles and Bromfenac/Levofloxacin Solution Improve Drug Corneal Penetration. International Journal of Nanomedicine 16, 5343-5356.
Primo, E. D., Otero, L. H., Ruiz, F., Klinke, S., Giordano, W., 2018. The disruptive effect of lysozyme on the bacterial cell wall explored by an in-silico structural outlook. Biochemistry and Molecular Biology Education 46, 83-90. https://doi.org/10.1002/bmb.21092
Razdan, K., Kanta, S., Chaudhary, E., Kumari, S., Rahi, D. K., Yadav, A. K., Sinha, V. R., 2023. Levofloxacin loaded clove oil nanoscale emulgel promotes wound healing in Pseudomonas aeruginosa biofilm infected burn wound in mice. Colloids and Surfaces B: Biointerfaces 222, 113113. https://doi.org/10.1016/j.colsurfb.2022.113113
Sarkar, S., Gulati, K., Mishra, A., Poluri, K. M., 2020. Protein nanocomposites: Special inferences to lysozyme based nanomaterials. International Journal of Biological Macromolecules 151, 467-482. https://doi.org/10.1016/j.ijbiomac.2020.02.179
Song, F., Chen, J., Zhang, Z., Tian, S., 2023. Preparation, characterization, and evaluation of flaxseed oil liposomes coated with chitosan and pea protein isolate hydrolysates. Food Chemistry 404, 134547. https://doi.org/10.1016/j.foodchem.2022.134547
Song, H., Inaka, K., Maenaka, K., Matsushima, M., 1994. Structural Changes of Active Site Cleft and Different Saccharide Binding Modes in Human Lysozyme Co-crystallized with Hexa-N-acetyl-chitohexaose at pH 4.0. Journal of Molecular Biology 244, 522-540. https://doi.org/10.1006/jmbi.1994.1750
Song, Y., Babiker, E. E., Usui, M., Saito, A., Kato, A., 2002. Emulsifying properties and bactericidal action of chitosan-lysozyme conjugates. Food Research International 35, 459-466. https://doi.org/10.1016/S0963-9969 (01) 00144-2
Tai, K., Rappolt, M., Mao, L., Gao, Y., Li, X., Yuan, F., 2020. The stabilization and release performances of curcumin-loaded liposomes coated by high and low molecular weight chitosan. Food Hydrocolloids 99, 105355. https://doi.org/10.1016/j.foodhyd.2019.105355
Tallian, C., Tegl, G., Quadlbauer, L., Vielnascher, R., Weinberger, S., Cremers, R., Pellis, A., Salari, J. W. O., Guebitz, G. M., 2019. Lysozyme-Responsive Spray-Dried Chitosan Particles for Early Detection of Wound Infection. ACS Appl. Bio Mater. 2, 1331-1339. https://doi.org/10.1021/acsabm.9b00023
Tan, C., Xue, J., Eric, K., Feng, B., Zhang, X., Xia, S., 2013. Dual Effects of Chitosan Decoration on the Liposomal Membrane Physicochemical Properties As Affected by Chitosan Concentration and Molecular Conformation. J. Agric. Food Chem. 61, 6901-6910. https://doi.org/10.1021/jf401556u
Tong, S. Y. C., Davis, J. S., Eichenberger, E., Holland, T. L., Fowler, V. G., 2015. Staphylococcus aureus infections: epidemiology, pathophysiology, clinical manifestations, and management. Clin Microbiol Rev 28, 603-661. https://doi.org/10.1128/CMR.00134-14
Tu, Q., Li, S., Zeng, Z., Liu, Y., Wang, C., Chen, S., Hu, B., Li, C., 2023. Cinnamon essential oil liposomes modified by sodium alginate-chitosan: application in chilled pork preservation. International Journal of Food Science & Technology 58, 939-953. https://doi.org/10.1111/ijfs.16140
Wang, Xingwei, Cheng, F., Wang, Xuejiao, Feng, T., Xia, S., Zhang, X., 2021. Chitosan decoration improves the rapid and long-term antibacterial activities of cinnamaldehyde-loaded liposomes. International Journal of Biological Macromolecules 168, 59-66. https://doi.org/10.1016/j.ijbiomac.2020.12.003
Wang, Y., Lv, Q., Chen, Y., Xu, L., Feng, M., Xiong, Z., Li, J., Ren, J., Liu, J., Liu, B., 2023. Bilayer hydrogel dressing with lysozyme-enhanced photothermal therapy for biofilm eradication and accelerated chronic wound repair. Acta Pharmaceutica Sinica B 13, 284-297. https://doi.org/10.1016/j.apsb.2022.03.024
Wu, X., Xin, M., Chen, H., Yang, L., Jiang, H., 2010. Novel mucoadhesive polysaccharide isolated from Bletilla striata improves the intraocular penetration and efficacy of levofloxacin in the topical treatment of experimental bacterial keratitis. Journal of Pharmacy and Pharmacology 62, 1152-1157. https://doi.org/10.1111/j.2042-7158.2010.01137.x
Yan, X., Gao, X., Li, X., Qiu, Q., Li, C., Yan, N., Li, J., Liu, M., Tang, X., Liu, X., Song, Y., Deng, Y., 2023. Persistent immune response: Twice tumor exfoliation induced by sialic acid-modified vincristine sulfate liposomes. International Journal of Pharmaceutics 631, 122467. https://doi.org/10.1016/j.ijpharm.2022.122467
Yang, X., Yang, K., Wu, S., Chen, X., Yu, F., Li, J., Ma, M., Zhu, Z., 2010. Cytotoxicity and wound healing properties of PVA/ws-chitosan/glycerol hydrogels made by irradiation followed by freeze-thawing. Radiation Physics and Chemistry 79, 606-611. https://doi.org/10.1016/j.radphyschem.2009.12.017
Zhang, L., Chen, D., Yu, D., Regenstein, J. M., Jiang, Q., Dong, J., Chen, W., Xia, W., 2022. Modulating physicochemical, antimicrobial and release properties of chitosan/zein bilayer films with curcumin/nisin-loaded pectin nanoparticles. Food Hydrocolloids 133, 107955. https://doi.org/10.1016/j.foodhyd.2022.107955
Zhong, T., Huang, R., Sui, S., Lian, Z., Sun, X., Wan, A., Li, H., 2015. Effects of ultrasound treatment on lipid self-association and properties of methylcellulose/stearic acid blending films. Carbohydrate Polymers 131, 415-423. https://doi.org/10.1016/j.carbpol.2015.06.026
Zhou, Q., Si, Z., Wang, K., Li, K., Hong, W., Zhang, Y., Li, P., 2022. Enzyme-triggered smart antimicrobial drug release systems against bacterial infections. Journal of Controlled Release 352, 507-526. https://doi.org/10.1016/j.jconrel.2022.10.038
Zhou, X., Zong, X., Wang, S., Yin, C., Gao, X., Xiong, G., Xu, X., Qi, J., Mei, L., 2021. Emulsified blend film based on konjac glucomannan/carrageenan/camellia oil: Physical, structural, and water barrier properties. Carbohydrate Polymers 251, 117100. https://doi.org/10.1016/j.carbpol.2020.117100

### Exemplary Embodiments

Embodiment 1. A composition, comprising: (a) a liposome coated with a polysaccharide, wherein the polysaccharide is biodegradable and biocompatible; and (b) an antibiotic encapsulated in the polysaccharide-coated liposome, wherein the polysaccharide is degradable by a lysozyme and/or forms a conjugate with the lysozyme.

Embodiment 2. The composition of Embodiment 1, wherein the polysaccharide comprises a deacetylated unit linked to an acetylated unit.

Embodiment 3. The composition of Embodiment 1 or Embodiment 2, wherein the polysaccharide comprises a D-glucosamine unit linked to an N-acetyl-D-glucosamine unit.

Embodiment 4. The composition of any one of Embodiments 1-3, wherein the polysaccharide comprises a chitosan.

Embodiment 5. The composition of any one of Embodiments 1-4, wherein the polysaccharide has a degree of deacetylation (DD) between about 50% and about 95%.

Embodiment 6. The composition of any one of Embodiments 1-5, wherein the polysaccharide is a chitosan having a degree of deacetylation (DD) of about 50%, about 55%, about 60%, about 65%, about 70%, about 75%, about 80%, about 85%, about 90%, and about 95%.

Embodiment 7. The composition of any one of Embodiments 1-6, wherein the polysaccharide is a chitosan having a degree of deacetylation (DD) of less than 75%, less than 60%, less than 65%, less than 60%, less than 55%, or less than 50%.

Embodiment 8. The composition of any one of Embodiments 1-7, wherein the polysaccharide is cross-linked.

Embodiment 9. The composition of any one of Embodiments 1-8, wherein the liposome comprises a lipid bilayer comprising: i) lecithin and cholesterol, or ii) cinnamon essential oil.

Embodiment 10. The composition of Embodiment 9, wherein the weight ratio of lecithin to cholesterol in the liposome is about 4:1.

Embodiment 11. The composition of Embodiment 9 or Embodiment 10, wherein the lecithin is soy-derived or egg-derived, and wherein the cholesterol is synthetic or of animal origin.

Embodiment 12. The composition of any one of Embodiments 1-11, wherein the liposome is prepared using a thin-film hydration method.

Embodiment 13. The composition of any one of Embodiments 1-12, wherein the composition comprises a plurality of polysaccharide-coated liposomes.

Embodiment 14. The composition of Embodiment 13, wherein the average particle size of the plurality of polysaccharide-coated liposomes is between about 150 nm and about 450 nm.

Embodiment 15. The composition of Embodiment 13 or Embodiment 14, wherein the average particle size of the plurality of polysaccharide-coated liposomes is at least about 1.5, at least about 2, or at least about 3 times the average particle size of a reference plurality of liposomes which are not coated with the polysaccharide.

Embodiment 16. The composition of any one of Embodiments 13-15, wherein the particle distribution index (PDI) of the plurality of polysaccharide-coated liposomes is between about 0.6 and about 0.8.

Embodiment 17. The composition of any one of Embodiments 13-16, wherein the particle distribution index (PDI) of the plurality of polysaccharide-coated liposomes is at least about 2, at least about 3, or at least about 4 times the PDI of a reference plurality of liposomes which are not coated with the polysaccharide.

Embodiment 18. The composition of any one of Embodiments 13-17, wherein the zeta potential of the plurality of polysaccharide-coated liposomes is between about 20 mV and about 40 mV.

Embodiment 19. The composition of any one of Embodiments 13-18, wherein the zeta potential of the plurality of polysaccharide-coated liposomes is positive and the zeta potential of a reference plurality of liposomes which are not coated with the polysaccharide is negative.

Embodiment 20. The composition of any one of Embodiments 1-19, wherein the antibiotic encapsulated in the polysaccharide-coated liposome is levofloxacin or vancomycin.

Embodiment 21. The composition of any one of Embodiments 1-20, wherein the antibiotic is present in a concentration of 1%-2% w/v in the total volume of the composition.

Embodiment 22. The composition of any one of Embodiments 1-21, wherein the antibiotic is of pharmaceutical grade with a purity of >98%.

Embodiment 23. The composition of any one of Embodiments 1-22, wherein the antibiotic is at least 5% by weight of the composition.

Embodiment 24. The composition of any one of Embodiments 1-23, wherein the antibiotic is a first antibiotic and the polysaccharide-coated liposome further encapsulates a second antibiotic different from the first antibiotic.

Embodiment 25. The composition of any one of Embodiments 1-24, wherein the polysaccharide-coated liposome encapsulates levofloxacin and vancomycin.

Embodiment 26. The composition of any one of Embodiments 1-25, further comprising a pharmaceutically acceptable carrier or excipient.

Embodiment 27. The composition of any one of Embodiments 1-26, further comprising a stabilizing agent.

Embodiment 28. The composition of Embodiment 27, wherein the stabilizing agent is selected from the group consisting of glycerol, propylene glycol, and polyethylene glycol.

Embodiment 29. The composition of Embodiment 27 or Embodiment 28, wherein the stabilizing agent is present in a concentration of 0.5% w/v in the total volume of the composition.

Embodiment 30. The composition of any one of Embodiments 1-29, wherein the polysaccharide-coated liposome comprises a targeting ligand.

Embodiment 31. The composition of Embodiment 30, wherein the targeting ligand is an antibody or epitope binding fragment thereof.

Embodiment 32. The composition of Embodiment 30 or Embodiment 31, wherein the targeting ligand specifically binds to Staphylococcus aureus.

Embodiment 33. The composition of any one of Embodiments 30-32, wherein the targeting ligand is on an outer surface of the polysaccharide-coated liposome.

Embodiment 34. The composition of any one of Embodiments 30-33, wherein the targeting ligand is conjugated to the polysaccharide coating of the polysaccharide-coated liposome.

Embodiment 35. The composition of any one of Embodiments 1-34, further comprising an anti-fouling agent.

Embodiment 36. The composition of any one of Embodiments 1-35, further comprising an agent that prevents non-specific protein adsorption.

Embodiment 37. The composition of any one of Embodiments 1-36, further comprising poly(ethylene glycol) (PEG).

Embodiment 38. The composition of any one of Embodiments 35-37, wherein the anti-fouling agent, the agent that prevents non-specific protein adsorption, and/or the PEG is on an outer surface of the polysaccharide-coated liposome.

Embodiment 39. The composition of any one of Embodiments 30-38, wherein the anti-fouling agent, the agent that prevents non-specific protein adsorption, and/or the PEG is conjugated to the polysaccharide coating of the polysaccharide-coated liposome.

Embodiment 40. The composition of any one of Embodiments 1-39, further comprising an antioxidant to protect the polysaccharide-coated liposome and/or the encapsulated antibiotic from degradation.

Embodiment 41. The composition of Embodiment 40, wherein the antioxidant is vitamin E.

Embodiment 42. The composition of any one of Embodiments 1-41, further comprising an enzyme inhibitor to regulate the degradation rate of the polysaccharide coating of the polysaccharide-coated liposome.

Embodiment 43. The composition of Embodiment 42, wherein the enzyme inhibitor is a lysozyme inhibitor.

Embodiment 44. The composition of any one of Embodiments 1-43, further comprising a fluorescence marker for imaging and tracking the distribution of the liposome, optionally wherein the fluorescence marker is fluorescein isothiocyanate (FITC).

Embodiment 45. The composition of any one of Embodiments 1-44, which is a suspension or colloid in water.

Embodiment 46. The composition of any one of Embodiments 1-44, which is a lyophilized composition.

Embodiment 47. The composition of Embodiment 46, further comprising a cryoprotectant added prior to and/or after lyophilization.

Embodiment 48. The composition of Embodiment 47, wherein the cryoprotectant is sucrose or trehalose.

Embodiment 49. The composition of any one of Embodiments 1-48, which is suitable for oral, ocular, topical, transdermal, subcutaneous, intradermal, oral, intranasal, intratracheal, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, or epicutaneous administration.

Embodiment 50. The composition of any one of Embodiments 1-49, which is encapsulated within a biodegradable polymer for controlled release.

Embodiment 51. The composition of Embodiment 50, wherein the biodegradable polymer is polylactic acid (PLA) or polyglycolic acid (PGA).

Embodiment 52. The composition of any one of Embodiments 1-51, which is formulated for delayed release of the antibiotic until the polysaccharide-coated liposome contacts a lysozyme.

Embodiment 53. The composition of any one of Embodiments 1-52, which is in contact with a biofilm.

Embodiment 54. The composition of Embodiment 53, wherein the biofilm is a biofilm of a gram-negative bacterium.

Embodiment 55. The composition of Embodiment 53 or Embodiment 54, wherein the biofilm is a biofilm of S. aureus.

Embodiment 56. The composition of any one of Embodiments 1-55 for use in treating a persistent bacterial infection in a subject in need thereof.

Embodiment 57. Use of the composition of any one of Embodiments 1-55 in treating a persistent bacterial infection in a subject in need thereof.

Embodiment 58. Use of the composition of any one of Embodiments 1-55 in the manufacture of a medicament for treating a persistent bacterial infection in a subject in need thereof.

Embodiment 59. A method of treating a persistent bacterial infection in a subject in need thereof, comprising administering an effective amount of the composition of any one of Embodiments 1-55 to the subject.

Embodiment 60. The method of Embodiment 59, wherein the persistent bacterial infection is S. aureus infection.

Embodiment 61. The method of Embodiment 59 or Embodiment 60, wherein the polysaccharide-coated liposome in the composition contacts a biofilm in the subject.

Embodiment 62. The method of Embodiment 61, wherein the polysaccharide coating of the polysaccharide-coated liposome is degraded by a lysozyme at the biofilm in the subject, thereby releasing the antibiotic at the biofilm.

Embodiment 63. The method of Embodiment 61 or Embodiment 62, wherein the polysaccharide coating of the polysaccharide-coated liposome forms a conjugate with the lysozyme at the biofilm in the subject.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method of treating a persistent bacterial infection in a subject in need thereof, comprising:
(a) administering an effective amount of a composition to the subject, wherein the composition comprises:
a polysaccharide-coated liposome comprising a liposome coated with a polysaccharide, wherein the polysaccharide is a chitosan having a degree of deacetylation (DD) of 70% or lower; and
an antibiotic encapsulated in the polysaccharide-coated liposome, wherein the antibiotic is levofloxacin; and
(b) at the site of a lysozyme in the subject, allowing the polysaccharide to be degradable by the lysozyme or form a conjugate with the lysozyme, thereby releasing the antibiotic at the site of the lysozyme to treat the persistent bacterial infection in the subject.

2. The method of claim 1, wherein the chitosan has a DD of about 50%.

3. The method of claim 1, wherein the chitosan has a DD of about 70%.

4. The method of claim 1, wherein the polysaccharide-coated liposome comprises a lipid bilayer comprising lecithin and cholesterol.

5. The method of claim 4, wherein the weight ratio of lecithin to cholesterol in the polysaccharide-coated liposome is about 4:1.

6. The method of claim 5, wherein the lecithin is soy-derived or egg-derived, and wherein the cholesterol is synthetic or of animal origin.

7. The method of claim 1, wherein the polysaccharide-coated liposome comprises a lipid bilayer comprising cinnamon essential oil.

8. The method of claim 1, wherein the polysaccharide-coated liposome is prepared using a thin-film hydration method.

9. The method of claim 1, wherein the composition comprises a plurality of polysaccharide-coated liposomes, and the average particle size of the plurality of polysaccharide-coated liposomes is between about 150 nm and about 450 nm.

10. The method of claim 9, wherein the average particle size of the plurality of polysaccharide-coated liposomes is at least about 1.5, at least about 2, or at least about 3 times the average particle size of a reference plurality of liposomes that are not coated with the polysaccharide.

11. The method of claim 9, wherein the particle distribution index (PDI) of the plurality of polysaccharide-coated liposomes is between about 0.6 and about 0.8.

12. The method of claim 9, wherein the particle distribution index (PDI) of the plurality of polysaccharide-coated liposomes is at least about 2, at least about 3, or at least about 4 times the PDI of a reference plurality of liposomes that are not coated with the polysaccharide.

13. The method of claim 9, wherein the zeta potential of the plurality of polysaccharide-coated liposomes is between about 20 mV and about 40 mV.

14. The method of claim 9, wherein the zeta potential of the plurality of polysaccharide-coated liposomes is positive and the zeta potential of a reference plurality of liposomes that are not coated with the polysaccharide is negative.

15. The method of claim 1, wherein the antibiotic is present in a concentration of 1%-2% w/v in the total volume of the composition, and the antibiotic is at least 5% by weight of the composition.

16. The method of claim 1, wherein the persistent bacterial infection is S. aureus infection.

17. The method of claim 1, wherein the polysaccharide-coated liposome in the composition contacts the lysozyme at a biofilm in the subject, and the polysaccharide coating of the polysaccharide-coated liposome is degraded by the lysozyme at the biofilm in the subject, thereby releasing the antibiotic at the biofilm.
